(19) United States
(12) Patent Application Publication
(10) Pub. No.: US 2007/0149616 A1
(43) Pub. Date: Jun. 28, 2007

(54) C3-Convertase Inhibitors for the Prevention and Treatment of Age-Related Macular Degeneration in Patients With At Risk Variants Of Complement Factor H

(75) Inventors: Abbot F. Clark, Arlington, TX (US); Allan R. Shepard, Fort Worth, TX (US)

Correspondence Address: ALCON, IP LEGAL, TB4-8, 6201 SOUTH FREEWAY, FORT WORTH, TX, 76134, US

(73) Assignee: Alcon Manufacturing, Ltd., Fort Worth, TX (US)

(21) Appl. No.: 11/614,595

(22) Filed: Dec. 21, 2006

Related U.S. Application Data
(60) Provisional application No. 60/753,135, filed on Dec. 22, 2005.

(51) Int. Cl. A61K 31/235; C12Q 1/68

(52) U.S. Cl. 514/533; 435/6

(57) ABSTRACT

The present invention provides methods for identifying a patient at risk for developing AMD by identifying the presence of the Y402H polymorphism or other at risk variants in the complement factor H gene. The present invention further provides methods for treating persons having AMD or at risk for developing AMD as a result of having the Y402H polymorphism or other at risk variants in the complement factor H gene.

### BACKGROUND OF THE INVENTION

This application claims priority to U.S. application Ser. No. 60/753,135, filed Dec. 22, 2005.

### FEILD OF THE INVENTION

The present invention relates to the field of prevention and treatment of ophthalmic diseases. More specifically, the present invention relates to the prevention and treatment of AMD in patients having the Y402H, or other at risk variants, of Complement Factor H (CFH) by administering agents that inhibit the conversion and activation of C3.

### DESCRIPTION OF THE RELATED ART

Age-related macular degeneration (AMD) is a debilitating, blinding disease that affects the macular or central area of the retina responsible for high-acuity vision and is the leading cause of irreversible vision loss in the elderly. Both genetic and environmental factors are known to play a role in the development of AMD. For example, smoking, lipid intake and age are known risk factors for the development of AMD. The two forms of AMD, dry-AMD and wet-AMD, affect more than 11 million individuals in the US. Dry-AMD occurs in 80% of AMD patients and is characterized by the presence of cellular debris (drusen) in Bruch's membrane under the retinal pigment epithelium (RPE), irregularities in the RPE pigmentation, or geographic atrophy. Wet-AMD, occurring in the remaining 20% of AMD patients, is characterized by choroidal neovascularization and/or detachment of the RPE. Extracellular matrix abnormalities in the eyes of AMD patients have also been implicated.

The diagnosis of dry age-related macular degeneration is defined by the presence of drusen under the RPE and is seen in the early stages of disease. Drusen are small yellowish extracellular deposits composed of protein, lipid, and cellular debris. A major component of Drusen are complement proteins. Drusen usually are confluent with significant pigment changes and accumulation of pigment in the posterior pole. RPE often appears atrophic with an easier visualization of the underlying choroidal plexus. In advanced stages of dry AMD, these focal islands of atrophy coalesce and form large zones of atrophy with severely affected vision. Wet AMD is defined by the presence of choroidal neovascularization and may include RPE elevation, exudate, or subretinal fluid.

There is currently no treatment to reverse the effects of AMD, however, the Age-Related Eye Disease Study (AREDS) showed that dietary antioxidant supplements may reduce the progression of AMD. [AREDS Report No. 8 (2001)] Laser photocoagulation, Photodynamic Therapy, Macugen® and Lucentis® are approved treatments available for wet-AMD. Macugen® is administered via intravitreal injection every six weeks, whereas Lucentis® is administered via intravitreal injection once a month. The active ingredient in Macugen®, pegaptanib sodium, is a covalent conjugate of an oligonucleotide, which is an antagonist of vascular endothelial growth factor (VEGF). The active ingredient in Lucentis®, ranibizumab, is an antibody fragment that binds VEGF. VEGF has been shown to be a key mediator of neovascularization associated with intraocular disorders (Ferrara et al. 1997). The concentration of VEGF in eye fluids are highly correlated to the presence of active proliferation of blood vessels in patients with diabetic and other ischemia-related retinopathies (Aiello et l. 1994). Other studies have demonstrated the localization of VEGF in choroidal neovascular membranes in patients affected by AMD (Lopez et al. 1996).

A large number of research groups have been intensively searching genes associated with and responsible for the development of AMD. Single nucleotide polymorphism (SNP) genotyping offers great promise in rapidly identifying disease associated genes (Hirschhorn & Daly, 2005; Hinds et al. 2005). Reports published in Science Express and PNAS (March-May, 2005) describe the use of SNP genotyping to identify a single polymorphism in the complement factor H gene (CFH) that accounts for elevated risk in developing AMD. A single amino acid change (Y402H) in CFH is reported to account for 40-50% of AMD.

The Edwards study (Edwards et al. 2005) involved scientists at UT Southwestern, Boston University and Sequenom. They performed SNP genotyping through the ARMD1 locus initially using 24 SNPs, then further refining the area with additional SNPs, in 2 case controlled populations (224 AMD patients and 134 controls in the first population; 176 cases & 68 controls in the second). They report that the individuals with one copy of the Y402H SNP in complement factor H had a 2.7× increased risk of developing AMD. This is single SNP appears to account for 50% of AMD in their populations.

The Haines study (Haines et al. 2005) was a collaborative study done at Vanderbilt University and Duke University. Similar to the Edwards study, Haines and colleagues SNP genotyped their 2 AMD populations across the ARMD1 locus. Their populations consisted of 182 AMD families and a case control population of 495 AMD patients and 185 controls. They initially used 44 SNPs to screen across the ARMD1 locus, then refined their search using additional SNPs. In their overall AMD population they found that patients heterozygous (bearing one copy) of the Y402H SNP in CFH had a 2.45 elevated risk for AMD, while homozygous individuals (having both copies of this SNP) had a 3.33 fold risk. The risk was even higher for those patients with neovascular (wet) AMD (3.45 in heterozygous and 5.57 in homozygous). They estimate that this SNP is responsible for 43% of AMD in their population.

The Klein study (Klein et al. 2005) involved scientists at Rockefeller University, Yale University, The National Eye Institute (NEI), and EMMES Corporation. Unlike the previous 2 studies, the Klein group performed a genome-wide SNP genotype screen of 96 AMD patients and 50 controls using >116,000 SNPs. All the individuals in this study were clinically well-defined from the AREDS study population. The Klein group independently mapped the AMD susceptibility locus to chromosome 1q (the same regions as ARMD1) and identified the Y402H SNP in CFH as the risk allele. Individuals bearing one copy of this allele (heterozygous) had a 4.6× elevated risk, while individuals bearing this SNP on both chromosomes (homozygous) had a 7.4× elevated risk for AMD.

The Hageman study (Hageman et al. 2005) included patients from the University of Iowa and Columbia University. They based their analysis of CFH on their previous studies that identified complement in the formation of Drusen and previous linkage analysis studies that identified the chromosomal locus lq25-32. The Hageman group analyzed 900 AMD patients and 400 matched controls for SNPs within the CFH gene. In addition to the Y402H variant identified in the previous publications, Hageman et al. identified other AMD risk variants, such as 162V, intervening sequences 1, 2, 6, and 10, A307A, and A473A.

Confirmation of the Edwards, Haines, Klein, and Hageman findings may be found in at least three follow-up studies by Conley et al. (2005), Zareparsi et al. (2005) and Souied et al. (2005). Conley et al. (2005) identified a significant association of the Y402H variant with AMD patients in 796 familial and 196 sporadic AMD cases relative to 120 unaffected, unrelated controls. Zareparsi et al., (2005) found that the T>C substitution in exon 9 (Y402H) was associated with AMD in their single center study population. Souied et al. (2005) extended the original findings of the Y402H polymorphism association with AMD in the North American populations to the European (French) AMD population. Souied et al. examined 60 sporadic and 81 familial AMD cases and found a significant association of the Y402H polymorphism with AMD relative to 91 healthy controls. Thus, it appears that the Y402H polymorphism association with AMD is a reproducible and generalized finding.

None of the previously described studies propose a treatment regimen for those patients identified as being at risk for developing AMD or for progressing from dry-AMD to wet-AMD due to the presence of the Y402H polymorphism. What is needed is a method for identifying patients at risk for developing AMD and providing a preventative treatment regimen for those patients. Also needed is a treatment regimen for inhibiting vision loss or improving visual acuity in those patients who have already been diagnosed with AMD and are found to possess the Y402H polymorphism or other at risk variants in complement family genes.

### SUMMARY OF THE INVENTION

The present invention overcomes these and other drawbacks of the prior art by providing a method for treating persons having AMD or at risk for developing AMD as a result of having the Y402H polymorphism or other at risk variants in the complement factor H gene. According to the methods of the invention, a patient is identified as having the Y402H polymorphism, or other at risk variants, in the complement factor H gene. The identification of the Y402H polymorphism, or other at risk variants, may be accomplished by obtaining tissue, such as by a cheek swab or blood sample, from the patient. The complement factor H gene is isolated from the tissue by means that are routine for the skilled artisan. The sequence for the gene isolated from the patient is compared with the sequence of the complement factor H gene not containing the Y402H polymorphism (also referred to as the “normal complement factor H gene” or “wild-type complement factor H gene”) to determine whether the Y402H polymorphism is present in the tissue sample taken from the patient. If the patient is identified as possessing the Y402H polymorphism, a composition comprising a C3-convertase inhibitor is administered to the patient to inhibit the loss of visual acuity associated with age-related macular degeneration (AMD) or to prevent the development of AMD in the patient. Thus, the method of the invention comprises the following steps:

a) identifying a Y402H polymorphism in a patient by
i) obtaining a tissue sample from the patient; and
ii) analyzing the tissue sample for the presence of the Y402H polymorphism, wherein the presence of the Y402H polymorphism indicates an incresed risk for the development of AMD or for the progression of dry-AMD to wet-AMD;

b) administering to a patient identified in step (a) above as possessing the Y402H polymorphism a therapeutically effective amount of a composition comprising a C3-convertase inhibitor.

The amount of C3-convertase inhibitor present in the composition of the invention will typically be from 0.01% to 10% percent by weight. In preferred aspects of the method of the invention, the C3-convertase inhibitor is compstatin.

While the compositions of the invention may be delivered by any known means of local ocular delivery, the preferred methods of administration of the composition will by by topical ocular delivery, posterior juxtascleral administration, intravitreal injection, subTenons administration, or by implant, either intravitreal or transscleral. Preferably, the composition of the invention will be administered by posterior juxtascleral administration or by sustained delivery device implanted intravitreally.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 provides an overview of the complement system, illustrating the classical, MB-Lectin, and alternative pathways.

### DETAILED DESCRIPTION PREFERRED EMBODIMENTS

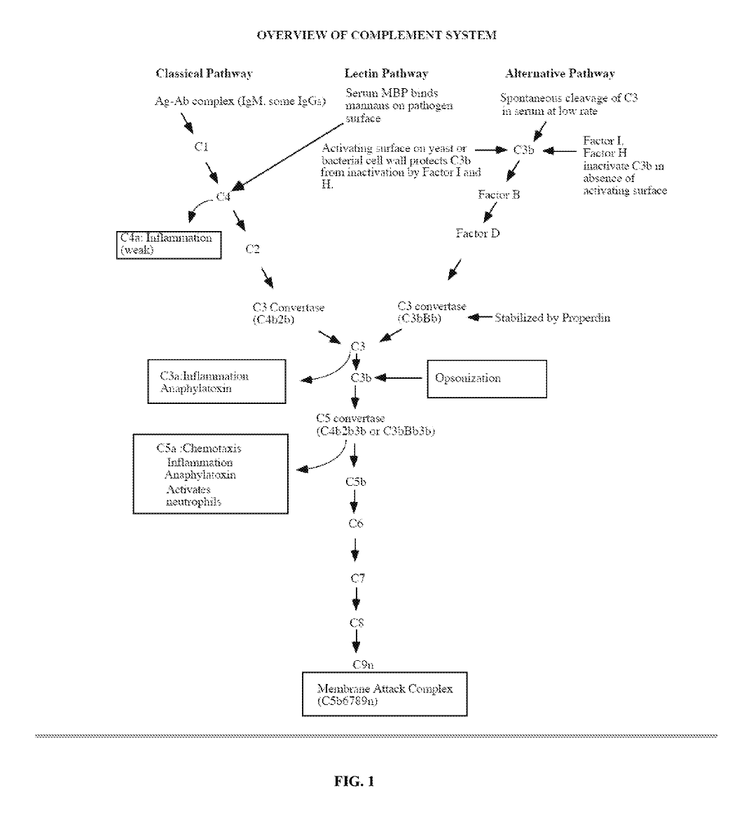

It has recently been reported that a single nucleotide polymorphism (SNP) in complement factor H (CFH) is responsible for nearly 50% of the attributable risk of AMD (Edwards et al., 2005; Haines et al. 2005; Klein et al. 2005; Hageman et al. 2005). The normal function of CFH appears to be to prevent excess complement activation. The complement system complements and amplifies the body's antibody response to foreign pathogens and is composed of three pathways: classical, MB-lectin, and alternative (FIG. 1). Activation of any of the complement cascades results in C3-convertase complex formation. Thus, C3-convertase is a central mediator of complement activation. C3-convertase converts C3 to its active C3b form. Classical/lectin pathways generate the C3-convertase C4b2b, whereas the alternative pathway generates the C3-convertase C3bBb. CFH is multifunctional and can bind C3b, accelerate C3b degradation, and act as a co-factor for factor I, which inactivates C3b. The Y402H SNP identified in the three recent studies lies in the S7 domain of the CFH protein, which is a region responsible for binding to C-Reactive Protein (CRP) and heparin. CFH bound to CRP and heparin increases the affinity of CFH for the complement protein C3b. Alteration of this interaction may occur with the Y402H polymorphism, resulting in reduced affinity for C3b and unchecked activation of the complement cascade.

The present invention relates to the prevention and treatment of AMD by inhibiting the conversion and activation of C3 using C3-convertase inhibitors. The target patient population of C3-convertase inhibitor therapy may be identified by genetic screening, e.g. using a cheek swab or blood analysis, and genotyping for the Y402H SNP or other at risk variants. Genomic DNA may be isolated from peripheral blood leukocytes using QIAamp DNA Blood Maxi Kits (Qiagen, Valencia, Calif.). DNA polymorphisms may be detected by single-strand conformational polymorphism (SSCP) analyses, using Applied Biosystems SNP Assays-On-Demand quantitative PCR, or by direct sequencing of amplified DNA. Other means of detecting polymorphisms in the CFH gene will be routine to the skilled artisan.

The C3-convertase inhibitors of the present invention can be administered either systemically or locally. Systemic administration includes: oral, transdermal, subdermal, intraperitioneal, subcutaneous, transnasal, sublingual, or rectal. The most preferred systemic route of administration is oral. Local administration for ocular administration includes: topical, intravitreal, periocular, transcleral, retrobulbar, juxtascleral, sub-tenon, or via an intraocular device. Preferred methods for local delivery include transscleral delivery to the macular by posterior juxtascleral administration; via intravitreal injection; or via cannula, such as that described in U.S. Pat. No. 6,413,245b1. Alternatively, the inhibitors may be delivered via a sustained delivery device implanted intravitreally or transsclerally, or by other known means of local ocular delivery.

The present invention is also directed to compositions containing C3-convertase inhibitors and analogs and methods for their use. According to the methods of the present invention, a composition comprising one or more compounds of the present invention and a pharmaceutically acceptable carrier for systemic or local administration is administered to a mammal in need thereof. Preferred compositions for use in the methods of the present invention contain a C3-convertase inhibitor, such as compstatin, rosmarinic acid, or MLN2222 (also known as CAB-2). The compositions are formulated in accordance with methods known in the art for the particular route of administration desired.

According to the methods of the present invention, a composition comprising one or more C3-convertase inhibitors and a pharmaceutically acceptable carrier for systemic or local administration is administered to a mammal in need thereof.

The compositions administered according to the present invention comprise a pharmaceutically effective amount of one or more C3-convertase inhibitors. As used herein, a “pharmaceutically effective amount” is one which is sufficient to reduce or prevent AMD and/or the loss of visual acuity associate with AMD. Generally, for compositions intended to be administered systemically for the treatment of AMD, the total amount of C3-convertase inhibitor will be about 0.01-100 mg/kg. For local administration, the preferred concentration of C3-convertase inhibitor in the composition will be from 0.0001% to 30% w/v.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Structure of Compstatin:

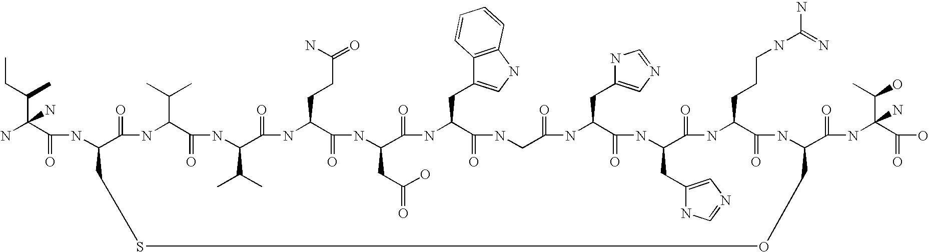

Linear Amino Acid Sequence of Compstatin:
ILE-CYS(2-12)-VAL-VAL-GLN-ASP-TRP-GLY-HIS-HIS-ARG-CYS(2-12)-THR
(Furlong et al. 2000; Morikis et al., 1998)

### EXAMPLE 2

Structure of Rosmarinic Acid: 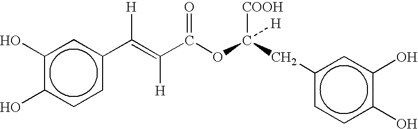
(Sahu et al., 1999)

### EXAMPLE 3

### MLN2222 from Millenium Pharmaceuticals (Formerly CAB-2)

Structure Unknown.

### EXAMPLE 4

This example illustrates the composition of a representative pharmaceutical formulation for intravitreal ophthalmic administration containing a C3-convertase inhibitor of the present invention.

| Ingredient | Amount (w/v, %) |
|---|---|
| C3-convertase inhibitor | 0.1-10 |
| PEG 400 | 10 |
| Polysorbate 80 | 0.5 |
| HPMC 2910 | 0.5 |
| Dibasic sodium phosphate, dodecahydrate | 0.18 |
| Sodium hydroxide | q.s. to pH |
| Hydrochloric acid | q.s. to pH |
| Water for Injection | q.s. to 100% |

### EXAMPLE 5

This example illustrates the composition of a representative pharmaceutical formulation containing a C3-convertase inhibitor of the invention, for posterior juxtascleral and periocular administration.

| Ingredients | Amount (w/v, %) |
|---|---|
| C3-convertase inhibitor | 5 |
| PEG 400 | 5 |
| Polysorbate 80 | 0.5 |
| HPMC 2910 | 0.5 |
| Dibasic sodium phosphate, dodecahydrate | 0.18 |
| Sodium Chloride | 0.17 |
| Sodium hydroxide | q.s. to pH |
| Hydrochloric acid | q.s. to pH |
| Water for Injection | q.s. to 100% |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents

U.S. Pat. No. 6,413,245b1

U.S. patent app 20030207309

Books

Other Publications
(Edwards et al., 2005; Hageman et al., 2005; Haines et al., 2005; Klein et al., 2005) Edwards, A. O., Ritter, R., 3rd, Abel, K. J., Manning, A., Panhuysen, C., Farrer, L. A., Haines, J. L., Hauser, M. A., Schmidt, S., Scott, W. K., et al. (2005). Complement factor H polymorphism and age-related macular degeneration Complement factor H variant increases the risk of age-related macular degeneration Strong association of the Y402H variant in complement factor H at lq32 with susceptibility to age-related macular degeneration. Science 308, 421-424.
Furlong, S. T., Dutta, A. S., Coath, M. M., Gormley, J. J., Hubbs, S. J., Lloyd, D., Mauger, R. C., Strimpler, A. M., Sylvester, M. A., Scott, C. W., and Edwards, P. D. (2000). C3 activation is inhibited by analogs of compstatin but not by serine protease inhibitors or peptidyl alpha-ketoheterocycles. Immunopharmacology 48, 199-212.
Hageman, G. S., Anderson, D. H., Johnson, L. V., Hancox, L. S., Taiber, A. J., Hardisty, L. I., Hageman, J. L., Stockman, H. A., Borchardt, J. D., Gehrs, K. M., et al. (2005). A common haplotype in the complement regulatory gene factor H (HFl/CFH) predisposes individuals to age-related macular degeneration. Proc Natl Acad Sci U S A 102, 7227-7232.
Haines, J. L., Hauser, M. A., Schmidt, S., Scott, W. K., Olson, L. M., Gallins, P., Spencer, K. L., Kwan, S. Y., Noureddine, M., Gilbert, J. R., et al. (2005). Complement factor H variant increases the risk of age-related macular degeneration. Science 308, 419-421.
Klein, R. J., Zeiss, C., Chew, E. Y., Tsai, J. Y., Sackler, R. S., Haynes, C., Henning, A. K., SanGiovanni, J. P., Mane, S. M., Mayne, S. T., et al. (2005). Complement factor H polymorphism in age-related macular degeneration. Science 308, 385-389.
Morikis, D., Assa-Munt, N., Sahu, A., and Lambris, J. D. (1998). Solution structure of Compstatin, a potent complement inhibitor. Protein Sci 7, 619-627.
Sahu, A., Rawal, N., and Pangbum, M. K. (1999). Inhibition of complement by covalent attachment of rosmarinic acid to activated C3b. Biochem Pharmacol 57, 1439-1446.

We claim:

1. A method for inhibiting the loss of visual acuity associated with age-related macular degeneration (AMD) in a patient having AMD or at risk for developing AMD due to the presence of a Y402H polymorphism or other at risk variant, said method comprising:
a) identifying said Y402H polymorphism or other at risk variant in said patient by
i) obtaining a tissue sample from said patient; and
ii) assaying said tissue sample for the presence of the Y402H polymorphism or other at risk variant, wherein the presence of the Y402H polymorphism or other at risk variant indicates an increased risk for the development of AMD or for the progression of dry-AMD to wet-AMD;
b) administering to a patient identified in step (a) above as possessing the Y402H polymorphism or other at risk variant a therapeutically effective amount of a composition comprising a C3-convertase inhibitor.

2. The method of claim 1, wherein the C3-convertase inhibitor is compstatin.

3. The method of claim 1, wherein the C3-convertase inhibitor is rosmarinic acid.

4. The method of claim 1, wherein the C3-convertase inhibitor is complement activation blocker-2 (CAB-2).

5. The method of claim 1, wherein the amount of C3-convertase inhibitor in the composition is from 0.01 to 10 percent by weight.

6. The method of claim 1, wherein the composition is administered via a method selected from the group consisting of oral administration, topical ocular administration, intravitreal injection, periocular administration, juxtascleral administration, retrobulbar administration, sub-tenon administration, transscleral, and via an intraocular device.

7. The method of claim 6, wherein the composition is administered by posterior juxtascleral administration.

8. The method of claim 7, wherein the composition is administered by sustained delivery device implanted intravitreally.
